(19) United States
(12) Patent Application Publication
(10) Pub. No.: US 2018/0159092 A1
(43) Pub. Date: Jun. 7, 2018

(54) BIOCOMPATIBLE COATED BATTERIES, SYSTEMS AND METHODS RELATED THERETO

(71) Applicant: Landsdowne Laboratories, Inc., Boston, MA (US)

(72) Inventors: Sumner A. Barenberg, Englewood, NJ (US); Jeffrey M. Karp, Brookline, MA (US); Bryan Laulicht, Danbury, CT (US)

(21) Appl. No.: 15/805,751

(22) Filed: Nov. 7, 2017

Related U.S. Application Data
(63) Continuation of application No. PCT/US16/31229, filed on May 6, 2016.
(60) Provisional application No. 62/158,241, filed on May 7, 2015.

(51) Int. Cl. H01M 2/02; H01M 2/34; H01M 10/04; A61L 31/18

(52) U.S. Cl. CPC H01M 2/026; H01M 2/345; H01M 2/0222; H01M 2220/30; H01M 10/0427; A61L 31/18; H01M 2200/20; H01M 2/0285

(57) ABSTRACT

This invention relates to a battery providing safety measures in case of ingestion. A battery may include a radiopaque marker distinguishing the battery from coins when x-rayed. A battery may include a pressure-sensitive coating that deactivates the battery in the absence of pressure.

### REFERENCE TO RELATED APPLICATIONS

This application is a continuation of Application No. PCT/US16/031229, filed May 6, 2016, which claims the benefit of U.S. Provisional Patent Application No. 62/158,241, filed May 7, 2015, the contents of each of which are fully incorporated by reference herein.

### BACKGROUND OF THE INVENTION

The widespread use of batteries to power many items including remote controls, flashlights, cameras, car key fobs, calculators, bathroom scales, reading lights, flameless candles, talking books, singing greeting cards, watches, thermometers, hearing aids, flashing jewelry, ornaments, games and toys creates an increased opportunity for ingestion. Children are particularly at risk to ingest batteries due to the accessibility and presence of these devices in the home. Recently, there was reported a nearly 7-fold increase in the percentage of reported button battery ingestions between 1985 and 2009.

Accidental ingestion has the strong potential for corrosive injury to the gastrointestinal tract with major complications, including esophageal burns, fistula, or perforation. Due to the electrochemistry, batteries retained in the esophagus may cause extensive damage including serious injuries and even death. While ingestion of batteries creates an immediate choking hazard, prolonged injury is due in large part to an electrical current from the battery that generates hydroxide ions through an electrolysis reaction that occurs when the battery is in contact with tissue fluids, such as saliva. A battery lodged in the esophagus or elsewhere in the digestive tract can cause serious injuries in as little as two hours. Moreover, such a battery may be mistaken for a coin when x-rayed, leading to delays in proper treatment. See also, Int. J. Pediatr. Otorhinolaryngol., 77(9): 1392-1399 (2013).

Batteries that are more readily identifiable as batteries when x-rayed and are less damaging when ingested are needed.

### SUMMARY OF INVENTION

In accordance with one or more embodiments, a battery that appears distinct from a coin when x-rayed is provided. The battery may comprise a conductive anode cap comprising an anode material, a cathode housing comprising a cathode material, a separator disposed between the anode material and the cathode material, a gasket joining the anode cap to the cathode housing, and a radiopaque marker, where the radiopaque marker generates a visible and distinctive or recognizable pattern on x-ray images of the battery that differs from the appearance of a coin on an x-ray image. In some implementations, the pattern includes one or more of a geometric pattern, letters, or other suitable other suitable designs that distinguish the battery from a coin when x-rayed.

In some implementations, the radiopaque marker is disposed on a plane parallel to the separator. In some implementations, the radiopaque marker is disposed on a plane perpendicular to the separator. In some implementations, the radiopaque marker comprises at least two loops encircling the anode cap and the conductive housing, wherein the at least two loops intersect at two points. The marker may be applied to an inner or outer surface of the anode cap and/or the cathode housing, or may be disposed on another element present in the battery interior.

In some implementations, the radiopaque marker comprises a material of greater radiodensity than the rest of the battery.

In some implementations, the radiopaque marker comprises a radiolucent material, wherein the pattern consists of a region of lower radiodensity than the surrounding battery.

In accordance with one or more embodiments, a battery with a built-in pressure-sensitive switch is provided. The battery may comprise a conductive anode cap comprising an anode material, a cathode housing comprising a cathode material, a separator disposed between the anode material and the cathode material, a gasket joining the anode cap to the cathode housing, and a pressure-sensitive coating covering the outer surfaces of the anode cap and the cathode housing. The pressure-sensitive coating exhibits a conductive state when a compressive stress above a compressive stress threshold is applied to the pressure-sensitive coating, and the compressive stress threshold is greater than a pre-determined applied stress associated with a digestive tract of a body. In some implementations, the pressure-sensitive coating comprises a radiopaque marker distinguishing the battery from a coin when x-rayed.

In some implementations, the pressure-sensitive coating is a quantum tunneling composite coating (QTCC). When a stress above the compressive stress threshold is applied to the QTCC, the QTCC is placed in a conductive state in which electrons are able to tunnel through the QTCC. Preferably, the QTCC is in a poorly-conductive or most preferably an insulating state when a stress below the compressive stress threshold is applied to the QTCC. The QTCC may comprise a polymer matrix with conductive microparticles suspended therein that collectively provide the pressure-sensitive conductive properties.

In certain embodiments, the compressive stress threshold is about or greater than 4 N/cm², about or greater than 15 N/cm², about or greater than 19 or 20 N/cm², or about or greater than 24 N/cm².

In certain implementations, the battery comprises an expansion layer that swells on wetting, wherein the expansion layer is disposed such that electrons are able to tunnel through the expansion layer when the expansion layer is dry, and wherein the expansion layer insulates the conductive anode cap and the cathode housing when wet. In some such implementations, the pressure-sensitive coating is the expansion layer.

In certain implementations, the battery is a button or a coin cell.

In certain implementations, the battery may include a partial or complete coating of a substance (for example, methylene blue) that changes the color of a subject's urine if ingested. In such implementations, a change in the color of a mammal's urine indicates that the mammal has ingested a battery even if the mammal is otherwise asymptomatic.

### BRIEF DESCRIPTION OF THE DRAWINGS

The batteries and methods described herein are set forth in the appended claims. However, for the purpose of explanation, several implementations are set forth in the following drawings:

FIGS. 1A and 1B are schematic views of a button battery with radiopaque markers, according to an illustrative implementation;

FIG. 2 is a schematic cross-sectional view of a button battery with a pressure-sensitive coating, according to an illustrative implementation; and

FIG. 3 is a schematic cross-sectional view of a button battery with a conductive coating, according to an illustrative implementation.

### DETAILED DESCRIPTION

In the following description, numerous details are set forth for the purpose of explanation. However, those of ordinary skill in the art will realize that the implementations described herein may be practiced without the use of these specific details and that the implementations described herein may be modified, supplemented, or otherwise altered without altering the scope of the batteries, systems, and methods described herein.

The batteries, systems, and methods described herein relate to batteries designed to reduce the risks associated with ingestion. A battery with a radiopaque marker that distinguishes the battery from a coin when x-rayed will be less likely to be misdiagnosed if ingested; a battery coated with a pressure-sensitive coating that acts as a switch may prevent the battery from hydrolyzing bodily fluids if ingested.

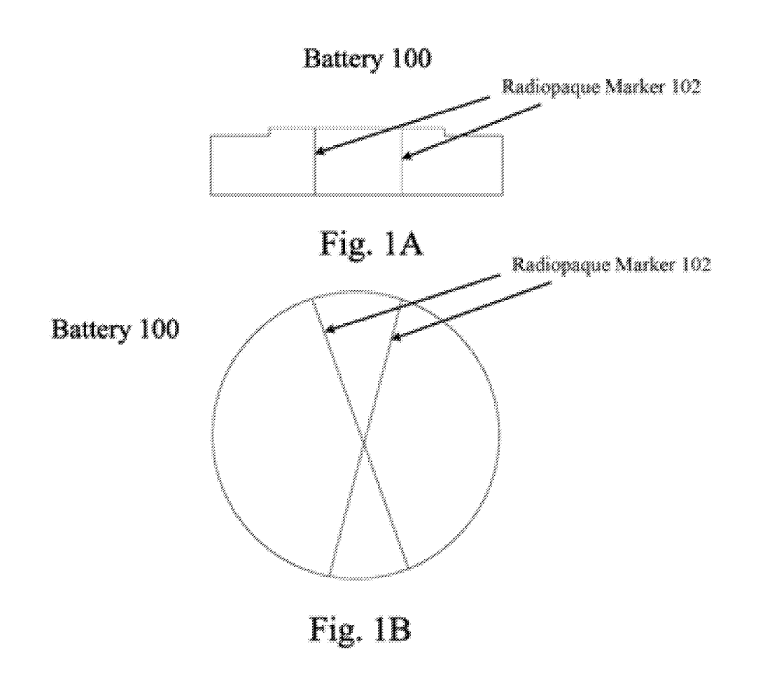

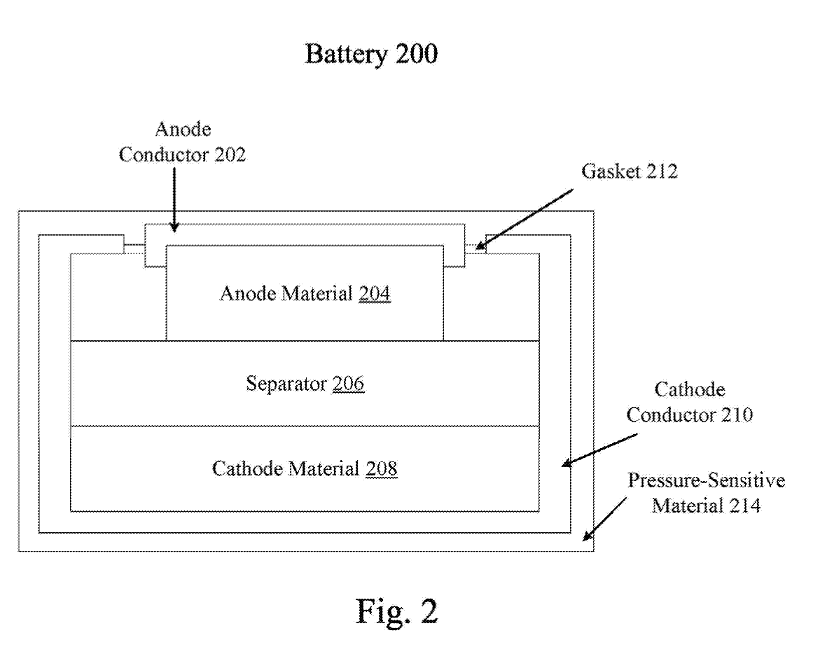

FIGS. 1A and 1B are schematic views of a button battery 100. FIG. 1A represents a profile view of battery 100, while FIG. 1B depicts a bottom-up view of battery 100. As described in relation to FIG. 2, battery 100 comprises a conductive anode cap and a cathode housing. The conductive anode cap comprises an anode conductor and an anode material; the cathode housing comprises a separator, a cathode material, a cathode conductor, and a seal or gasket holding the anode cap to the cathode housing. The seal electrically insulates the anode cap from the cathode housing. The electrolyte-soaked separator creates a barrier between the cathode and anode, preventing them from touching while allowing electrical charge to flow freely between them. When a load completes the circuit across the conductive anode cap and the cathode housing, the battery produces electricity through a series of electrochemical reactions between the anode, cathode and electrolytes. Battery 100 further comprises radiopaque markers 102. Radiopaque markers 102 are markers with a radiopacity differing from the radiopacity of the rest of battery 100, and thus distinguish battery 100 from a coin in an x-ray image. Radiopaque markers 102 may have a greater radiopacity than the rest of battery 100, or, in some implementations, may be more radiolucent than the rest of battery 100, so long as the markers 102 create a noticeable pattern when x-rayed.

As depicted, markers 102 comprise two loops surrounding the battery and intersecting at two points; creating a distinctive pattern regardless of the angle of viewing of the battery. In some implementations, markers 102 may take a different form. As illustrative examples, markers 102 may spell the word “battery” on both the top and the side of battery 100; may create a zig-zag pattern along the side of battery 100 and a “+” pattern along the anode cap of battery 100; or may take some other suitable form that will distinguish battery 100 from a coin in an x-ray image.

In certain embodiments, the radiopaque marker comprises a radiopaque material disposed in or on the battery, e.g., presenting a distinctive shape, sign, or pattern. For example, a marking may be placed on the anode cap, the cathode housing, or any other suitable part of the housing, to allow a treating physician to ascertain via X-ray imaging whether the type of battery swallowed is one according to this invention, i.e., presents a less urgent medical issue than a conventional-type battery, or simply to distinguish a battery from a similarly-shaped object such as a coin, particularly when viewed face-on (as opposed to edgewise, where the crimped shape of a coin battery itself is distinctive). In certain embodiments, the radiopaque marker may shaped so as to permit a treating physician to more readily recognize mark, e.g., the double ring or “halo” sign, as distinctive of a battery as compared to a coin when both are viewed on an x-ray image. In certain embodiments, the radiopaque material may be disposed in the gasket. Radiopaque material is any material applied that is not transparent to X-rays or other forms of radiation and can be distinguished from the material that forms the anode cap and/or the cathode housing in an X-ray image. Examples of radiopaque materials include, but are not limited to, tungsten, tungsten dioxide, tungsten trioxide, stainless steel powder, silver iodide, iodinated organic compounds, gold, nickel alloys, titanium, tantalum, iodine and barium, and salts thereof and radiopaque polymers. In certain embodiments, the radiopaque marker may be defined by altering the radiopacity of the battery, e.g., by etching (e.g., via laser or chemically) or by machining away material from the cathode housing or anode cap. In certain embodiments, the radiopaque material (e.g., a sheet, a sphere, or any other two- or three-dimensional object) may be located within the battery, e.g., disposed between the anode cap and the cathode housing.

FIG. 2 depicts a pressure-sensitive battery 200, which in some implementations may correspond to the battery 100 depicted in FIGS. 1A and 1B. As depicted, battery 200 comprises a conductive anode cap and a cathode housing. The conductive anode cap comprises an anode conductor 202 and an anode material 204; the cathode housing comprises a separator 206, a cathode material 208, a cathode conductor 210, and a gasket 212. As in a standard battery, when a load completes the circuit across the conductive anode cap and the cathode housing, the battery produces electricity through a series of electrochemical reactions between the anode, cathode, and electrolytes. Battery 200 further comprises a pressure-sensitive coating 214 covering the entire battery 200. Pressure-sensitive coating 214 is placed in a conductive state when it is subject to a compressive stress above a compressive stress threshold, but is otherwise nonconductive. The compressive stress threshold is greater than a pre-determined applied stress associated with a digestive tract of a body. In this way, the battery may function when placed in a device, but remain inactive in a lower-pressure environment such as a mammalian digestive tract.

Anode conductor 202 and cathode conductor 210 are conductive materials, and may include a metal, a polymer, or some other suitable material. Anode material 204 may be a lithium compound or some other suitable anode material. Cathode material 208 may be manganese dioxide or some other suitable cathode material. Separator 206 is a permeable membrane that permits the transport of ionic charge carriers, and may comprise electrolyte-soaked fibers, a polymer film, or some other suitable barrier. Gasket 212 may be an electrically insulating ring forming a seal on part of the anode material 204 and the cathode material 208, and may be a polymer.

In certain embodiments, the amount of pressure needed to activate battery 200 is a pressure that is typically applied to the anode cap when a battery is inserted into a battery-operated device. Such a device will typically have a battery compartment used to house the battery. When the battery is placed into the device, a sufficient force is applied to activate the battery and to operate the device. Devices that require a battery to operate include but are not limited to remote controls, flashlights, cameras, car key fobs, calculators, bathroom scales, reading lights, flameless candles, talking books, singing greeting cards, watches, thermometers, hearing aids, flashing jewelry, ornaments, games, laser pointers and toys. As the pressure applied by the device is normal and localized to the anode cap, only those portions of pressure-sensitive coating 214 directly under pressure will be placed in a conductive state—no short-circuit current will be generated.

To avoid harm if battery 200 is ingested, the pressure needed to activate battery 200 (i.e., place pressure-sensitive coating 214 in a conductive state) is greater than the pressure or stress applied by the digestive tract of a mammal. Since the digestive tract does not apply the stress or pressure needed to shift the pressure-sensitive coating 214 into a conductive state, a conductive pathway is not available between the anode cap and the electrolytic cell when a battery is ingested. Absent a conductive pathway, damage by the battery to the intestinal tissues is avoided. In humans, for example, the force needed activate the battery (i.e., shift the anode cap to a second position in which the anode cap is in electrical communication with the electrolytic cell) is greater than about 4 N/cm². And in certain embodiments, the force needed is greater than about 15 N/cm². And in certain embodiments, the force needed is greater than about 24.2 N/cm². For batteries at risk of ingestion by canines, the force needed activate the battery may be greater than about 19 N/cm².

Pressure-sensitive coating 214 may be a quantum tunneling composite coating (QTCC), comprising conductive microparticles suspended in a polymer matrix. The conductive microparticles may be silver, gold, carbon, or any other suitable conductive material; the polymer matrix may be an insulating PDMS or other suitable insulating and elastically deformable material. When the QTCC is unstressed, the conductive microparticles are spaced sufficiently far apart to prevent conduction, whether directly or via quantum tunneling of electrons. But when a stress greater than a compressive stress threshold is applied to the QTCC, the polymer matrix is deformed such that quantum tunneling between conductive microparticles and through the polymer matrix is enabled: the QTCC thus acts as a pressure-sensitive switch. The conductive microparticles may include a surface with a nanoscale roughness that enhances an electric field gradient such that, when the conductive microparticles are less than 1-5 nm apart, electrons are able to tunnel through the polymer matrix, thereby conducting current there through.

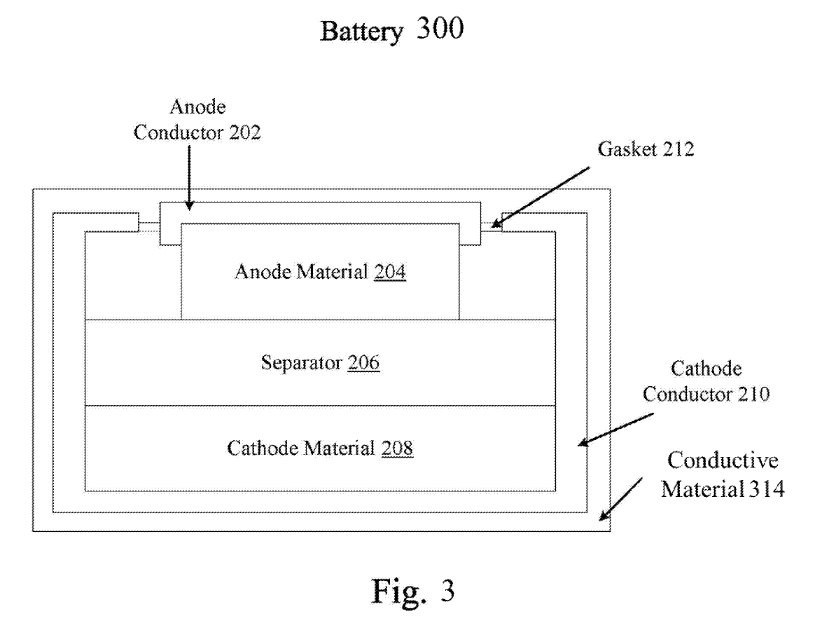

FIG. 3 depicts a battery 300 coated with a conductive material 314, which in some implementations may correspond to the battery 100 depicted in FIGS. 1A and 1B. As depicted, battery 300 comprises a conductive anode cap and a cathode housing. The conductive anode cap comprises an anode conductor 202 and an anode material 204; the cathode housing comprises a separator 206, a cathode material 208, a cathode conductor 210, and a gasket 212. As in a standard battery, when a load completes the circuit across the conductive anode cap and the cathode housing, the battery produces electricity through a series of electrochemical reactions between the anode, cathode, and electrolytes. Battery 300 further comprises a conductive material 314 covering the entire battery 300. While not intending to be bound by theory, the conductive material may limit or inhibit metal dissolution or erosion from the anode cap or cathode housing. For example, in certain embodiments the anode cap or cathode housing may comprise nickel. The conductive material would minimize or prevent leaching of nickel ions from the anode cap or cathode housing when a battery according to this invention is placed in an aqueous solution.

In certain embodiments, the conductive material 314 comprises a conductive polymer. Examples of conductive polymers include, but are not limited to, polypyrrole, polyaniline, polyacetylene, polythiophene, polyphenylene vinylene, polyphenylene sulfide, poly p-phenylene, and polyheterocycle vinylene.

In certain embodiments, the conductive material may also comprise a metal chelating agent. For example, the metal chelating agent may be disposed in the gasket or on an external surface of the battery. By way of non-limiting example, suitable chelating agents include aconitic acid, alanine diacetic acid (ADA), alkoyl ethylene diamine triacetic acids (e.g., lauroyl ethylene diamine triacetic acids (LED3A)), aminotri(methylenephosphonic acid) (ATMP), aspartic acid diacetic acid (ASDA), aspartic-N-monoacetic acid, diamino cyclohexane tetraacetic acid (CDTA), citraconic acid, citric acid, 1,2-diaminopropanetetraacetic acid (DPTA-OH), 1,3-diamino-2-propanoltetraacetic acid (DTPA), diethanolamine, diethanol glycine (DEG), diethylenetriaminepentaacetic acid (DTPA), diethylene triamine pentamethylene phosphonic acid (DTPMP), diglycolic acid, dipicolinic acid (DPA), ethanolaminediacetic acid, ethanoldiglycine (EDG), ethionine, ethylenediamine (EDA), ethylenediaminediglutaric acid (EDDG), ethylenediaminedi(hydroxyphenylacetic acid (EDDHA), ethylenediaminedipropionic acid (EDDP), ethylenediaminedisuccinate (EDDS), ethylenediaminemonosuccinic acid (EDMS), ethylenediaminetetraacetic acid (EDTA), ethylenediaminetetrapropionic acid (EDTP), ethyleneglycolaminoethylestertetraacetic acid (EGTA), gallic acid, glucoheptonic acid, gluconic acid, glutamic acid diacetic acid (GLDA), glutaric acid, glyceryliminodiacetic acid, glycinamidedisuccinic acid (GADS), glycoletherdiaminetetraacetic acid (GEDTA), 2-hydroxyethyldiacetic acid, hydroxyethylenediaminetriacetic acid (HEDTA), hydroxyethyldiphosphonic acid (HEDP), 2-hydroxyethyl imino diacetic acid (HIMDA), hydroxyiminodiacetic acid (HIDA), 2-hydroxy propylene diamine disuccinic acid (HPDDS), iminodiacetic acid (IDA), iminodisuccinic acid (IDS), itaconic acid, lauroyl ethylene diamine triacetic acids (LED3A), malic acid, malonic acid, methylglycinediacetate (MGDA), methyliminodiacetic acid (MIDA), monoethanolamine, nitrilotriacetic acid (NTA), nitrilotripropionic acid (NPA), N-phosphonomethyl glycine (glyphosate), propyldiamine tetraacetic acid (PDTA), salicylic acid, serinediacetic acid (SDA), sorbic acid, succinic acid, sugars, tartaric acid, tartronic acid, triethanolamine, triethylenetetraamine, triethylene tetraamine hexaacetic acid (TTHA), and combinations thereof.

Referring to FIGS. 1A and 1B, in some implementations, pressure-sensitive coating 214 may act as a radiopaque marker 102. As an illustrative example, if pressure-sensitive coating 214 is a QTCC, the polymer portion of QTCC may be radiolucent while the conductive microparticles may be radiopaque, and may thus create a distinctive pattern of dots surrounding battery 200 that would distinguish it from a coin when x-rayed.

In some implementations, pressure-sensitive coating 214 may include or be surrounded by an expansion layer that swells on wetting. Such an expansion layer may be an insulating layer, that, when dry, is thin enough to allow quantum tunneling by electrons to battery 200, but, when wet, swells to a size that prevents quantum tunneling by electrons through its bulk, and thus insulates battery 200 when placed in a wet environment. In some such implementations, the expansion layer may further displace conductive microparticles from the rest of battery 200, and thus create a clearer distinction between battery 200 and a coin in an x-ray image.

In certain embodiments, the gasket may comprise nylon, polyethylene, chlorotrifluoroethylene, fluorinated ethylene-propylene, polytetrafluoroethylene, perfluoroalkoxy polymer, polypropylene, polystyrene, polysulfone, polyvinyls, doped ionomers (such as Surlyn® (poly(ethylene-co-methacrylic acid), sodium salt)), polyphosphazenes, polyacrylonitrile and combinations thereof.

In certain embodiments, the battery may comprise an indicator element that when exposed to water changes color, e.g., disposed in the water-permeable gasket or on an exterior surface of the battery. In certain embodiments, the indicator element may be water-soluble such that it is released from the battery when the battery contacts water. In certain embodiments, the indicator element may be capable of leaching from the battery when the battery is in an aqueous environment and that, when ingested by a mammal, dyes the urine of the mammal a distinctive (e.g., non-yellow) color. Examples of indicator elements include, but are not limited to, Yellow No. 5, (β-carotene, rifampin, Yellow No. 6, tetracycline, Red No. 40, Red No. 3, Blue No. 2, Evan's Blue, Green No 3, Blue No. 1, methylene blue, indocyanine green, Betanin, and beet juice (or anthocyanines or other food-based dyes), and combinations thereof.

In certain embodiments, the battery further comprises an aversive agent. For example, the aversive agent may be disposed in the gasket or in the release element or on an external surface of the battery. Various aversive agents can be employed including, for example and without limitation, natural, artificial and synthetic flavor oils and flavoring aromatics and/or oils, oleoresins and extracts derived from plants, leaves, flowers, fruits, and so forth, and combinations thereof. Nonlimiting representative flavor oils include spearmint oil, peppermint oil, eucalyptus oil, oil of nutmeg, allspice, mace, oil of bitter almonds, menthol and the like. Also useful aversive agents are artificial, natural and synthetic fruit flavors such as citrus oils including lemon, orange, lime, grapefruit, and fruit essences and so forth. Additional aversive agents include sucrose derivatives (e.g., sucrose octaacetate), chlorosucrose derivatives, quinine sulphate, and the like. Additional aversive agents that may have pungent properties include but are not limited to capsaicin, piperine, allyl isothiocyanate, and resinferatoxin. An exemplary commercially available aversive agent includes Denatonium Benzoate NF-Anhydrous, sold under the name Bitterant-b, BITTER+PLUS, Aversion, or Bitrex™ (Macfarlan Smith Limited, Edinburgh, UK)

In certain embodiments, the battery may have an aesthetically unappealing appearance. For example, the anode cap and/or the cathode housing may have a dull, dark (e.g., gray, black) color. In certain embodiments, the battery may have a non-glossy or matte finish.

Preferred embodiments of this invention are described herein with reference to the drawings. Of course, variations, changes, modifications and substitution of equivalents of those preferred embodiments will become apparent to those of ordinary skill in the art upon reading the foregoing description. The inventors expect skilled artisans to employ such variations, changes, modifications and substitution of equivalents as appropriate, and the inventors intend for the invention to be practiced otherwise than specifically described herein. Those of skill in the art will readily recognize a variety of non-critical parameters that could be changed, altered or modified to yield essentially similar results. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. For example, a button battery may include a partial coating of methylene blue and no other feature, and thereby signal that it has been swallowed only through a change in the color of the urine of the mammal that ingested the battery; and a battery with a pressure-sensitive layer need not include a radiopaque marker.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A battery comprising:
a conductive anode cap;
a cathode housing;
an electrochemical cell comprising an anode material, a cathode material, and a separator disposed between the anode material and the cathode material;
a gasket joining the anode cap to the cathode housing; and
a radiopaque marker, wherein the radiopaque marker generates a pattern on x-ray images of the battery.

2. The battery of claim 1, wherein the pattern is a geometric pattern.

3. The battery of claim 1, wherein the pattern comprises letters.

4. The battery of claim 1, wherein the radiopaque marker is disposed on a plane parallel to the separator.

5. The battery of claim 1, wherein the radiopaque marker is disposed on a plane perpendicular to the separator.

6. The battery of claim 1, wherein the radiopaque marker comprises at least two loops encircling the anode cap and the conductive housing, wherein the at least two loops intersect at two points.

7. The battery of claim 1, wherein the radiopaque marker comprises a material of greater radiodensity than the rest of the battery.

8. The battery of claim 1, wherein the radiopaque marker comprises a radiolucent material, wherein the pattern consists of a region of lower radiodensity than the surrounding battery.

9. The battery according to claim 1, further comprising:
a pressure-sensitive coating covering the anode cap and the cathode housing, wherein the pressure-sensitive coating is placed in a conductive state when a compressive stress above a compressive stress threshold is applied to the pressure-sensitive coating,
wherein the compressive stress threshold is greater than a pre-determined applied stress associated with a digestive tract of a body.

10. The battery of claim 9, wherein the pressure-sensitive coating comprises the radiopaque marker.

11. The battery according to claim 10, wherein:
the pressure-sensitive coating is a quantum tunneling composite coating (QTCC), and when a stress above the compressive stress threshold is applied to the QTCC, the QTCC is placed in a conductive state in which electrons are able to tunnel through the QTCC.

12. The battery according to claim 11, wherein, when a stress below the compressive stress threshold is applied to the QTCC, the QTCC is in an insulating state.

13. The battery according to claim 12, wherein the QTCC comprises a polymer matrix with conductive microparticles suspended therein that collectively provide the pressure-sensitive conductive properties.

14. The battery according to claim 13, wherein the compressive stress threshold is about or greater than 15 N/cm².

15. The battery according to claim 14, wherein the compressive stress threshold is about or greater than 19 or 20 N/cm².

16. The battery according to claim 14, wherein the compressive stress threshold is about or greater than 24 N/cm².

17. The battery according to claim 16, further comprising an expansion layer, wherein:
the expansion layer swells on wetting;
the expansion layer is disposed such that electrons are able to tunnel through the expansion layer when the expansion layer is dry; and
the expansion layer is disposed such that the expansion layer insulates the conductive anode cap and the cathode housing when the expansion layer is wet.

18. The battery according to claim 17, wherein the pressure-sensitive coating is the expansion layer.

19. A battery comprising:
a conductive anode cap;
a cathode housing;
an electrochemical cell comprising an anode material, a cathode material, and a separator disposed between the anode material and the cathode material;
a gasket joining the anode cap to the cathode housing; and
a pressure-sensitive coating covering the anode cap and the cathode housing, wherein the pressure-sensitive coating is placed in a conductive state when a compressive stress above a compressive stress threshold is applied to the pressure-sensitive coating,
wherein the compressive stress threshold is greater than a pre-determined applied stress associated with a digestive tract of a body.

20. The battery according to claim 19, wherein:
the pressure-sensitive coating is a quantum tunneling composite coating (QTCC), and when a stress above the compressive stress threshold is applied to the QTCC, the QTCC is placed in a conductive state in which electrons are able to tunnel through the QTCC.

21. The battery according to claim 20, wherein, when a stress below the compressive stress threshold is applied to the QTCC, the QTCC is in an insulating state.

22. The battery according to claim 21, wherein the QTCC comprises a polymer matrix with conductive microparticles suspended therein that collectively provide the pressure-sensitive conductive properties.

23. The battery according to claim 22, wherein the compressive stress threshold is about or greater than 4 N/cm².

24. The battery according to claim 23, wherein the compressive stress threshold is about or greater than 15 N/cm²

25. The battery according to claim 23, wherein the compressive stress threshold is about or greater than 19 or 20 N/cm².

26. The battery according to claim 23, wherein the compressive stress threshold is about or greater than 24 N/cm².

27. The battery according to claim 26, wherein the expansion layer comprises a metal chelating agent

28-42. (canceled)
